(19) United States
(12) Patent Application Publication
(10) Pub. No.: US 2014/0350524 A1
(43) Pub. Date: Nov. 27, 2014

(54) Wire Guide Engagement And Withdrawal Tool And Method

(71) Applicant: COOK MEDICAL TECHNOLOGIES LLC, Bloomington, IN (US)

(72) Inventors: Therese J. O'Day, Bloomington, IN (US); Logan Cage, Bloomington, IN (US); James Elsesser, Bloomington, IN (US)

(73) Assignee: COOK MEDICAL TECHNOLOGIES LLC, Bloomington, IN (US)

(21) Appl. No.: 14/369,746

(22) PCT Filed: Dec. 18, 2012
(86) PCT No.: PCT/US2012/070284

§ 371 Date: Jun. 30, 2014

Related U.S. Application Data
(60) Provisional application No. 61/590,854, filed on Jan. 26, 2012.

(51) Int. Cl. A61M 25/09; A61M 25/01

(52) U.S. Cl. CPC A61M 25/09; A61M 25/0108
USPC 604/509; 604/171

(57) ABSTRACT

A tool for engaging and withdrawing a wire guide from a patient includes a deformable distal segment having a self-expanding bias such that the distal segment assumes a tortuous configuration, and a control line attached to the distal segment and configured to extend through an intraluminal sheath, such that pulling the control line deforms the distal segment in opposition to the self-expanding bias to tighten about a wire guide. The tool may be withdrawn from the patient while tightened about the wire guide, such that the wire guide simultaneously extends out of the patient at each of a first and a second percutaneous entry point.

### RELATION TO OTHER PATENT APPLICATION

This application claims priority to provisional patent application 61/590,854 filed Jan. 26, 2012, with the same title and is a United States National Stage of International Application PCT/US2012/07024.

### TECHNICAL FIELD

The present disclosure relates generally to engaging a wire guide within a body lumen of a patient, and relates more particularly to engaging a wire guide with a deformable tool via pulling an attached control line.

### BACKGROUND

Angioplasty, stenting and other techniques are well known practices for treating obstructed vessels within the human anatomy. In a conventional approach, a catheter is advanced through an entry point in the patient's skin and slid over a wire guide to a desired location within the patient's vasculature. The balloon, stent, or other treatment device may be placed within or near an obstruction in the vessel of interest, and then used to increase or restore blood flow. Various techniques have been used with great success for decades. As with other forms of peripheral intervention, clinicians continue to seek the capability to treat smaller vessels and those located in more difficult to access places within the human body.

Advancements in medical device technology have allowed treatment devices to traverse relatively great lengths within the body and reach constrictions within especially small vessels. Approaching a treatment location may be of little use, however, unless the associated wire guide over which a treatment device travels is able to successfully cross a constriction to enable advancing the treatment device into or past the constriction. Those skilled in the art will be familiar with the relative difficulty of pushing a wire guide through material of a lesion blocking a vein or artery in many instances. In the case of treating infrapopliteal arteries, for instance, matters may be further complicated by the location and nature of the disease. A significant challenge for a treating physician can be crossing constricted areas in these vessels from a vascular access site that is relatively far away. In one conventional approach, a sheath is inserted retrograde to blood flow in the femoral artery in the leg opposite the one to be treated. The sheath and the wire guide are navigated up through the iliac, and then steered down into the opposite leg. The wire guide may eventually be advanced past the sheath through the diseased vessel of interest, such as the popliteal artery, the anterior tibial, posterior tibial or peroneal artery. Crossing lesions in the diseased vessel from such a distance access point may be quite difficult. Each twist and turn through the tortuous path navigated just to reach the diseased vessel can reduce pushability of the wire guide. Moreover, should the diseased vessel have a chronic total occlusion, the wire guide may need to punch through a fibrous thrombus cap within the lesion. These fibrous caps may be calcified and especially difficult to puncture given the conventional wire guide's atraumatic distal tip.

Alternative approaches attempt to access the vessel to be treated through the same leg femoral artery, anterograde to blood flow. This strategy enables a relatively straight approach and shorter distance to the lesion to be treated, however, the external anatomy of the patient may not be conducive to this type of technique. Moreover, while force transmission through the wire guide and steering may be easier, the challenge of crossing a fibrous thrombus cap within the lesion may not be significantly diminished.

A relatively newer technique for crossing challenging lesions involves accessing the diseased artery from the ankle or foot and traversing the lesion retrograde to blood flow. A wire guide introduced in this manner may be more readily capable of puncturing a fibrous cap within the lesion. As an alternative to puncturing the fibrous cap, the wire guide is sometimes taken subintimally and then reenters the vessel on the other side of the lesion. In either case, if the wire guide successfully crosses the lesion, it can be captured with a snare placed above the lesion, i.e. upstream, and then pulled out from the patient's body at an upstream entry point. Snaring the wire guide, however, is by no means certain using conventional techniques. Moreover, conventional snaring devices even theoretically capable of grabbing a wire guide under such circumstances tend to be complex and expensive.

### SUMMARY OF THE DISCLOSURE

In one aspect, a tool for engaging and withdrawing a wire guide from a patient includes an elongate body having a deformable distal segment, and a rigid proximal segment for sliding the distal segment into and out of an intraluminal sheath. The distal segment includes a self-expanding bias such that the distal segment assumes a tortuous configuration defining a spatial envelope, in response to sliding out of the intraluminal sheath. The tool further includes a control line attached to the distal segment and configured to extend in parallel with the proximal segment through the intraluminal sheath, such that pulling the control line in a proximal direction tightens the distal segment in opposition to the self-expanding bias to engage a wire guide positioned within the spatial envelope.

In another aspect, a method of treating a patient includes advancing a wire guide through a body lumen of the patient from a first percutaneous entry point, and receiving an end of the wire guide within a spatial envelope defined by a tortuous wire guide engagement and withdrawal tool. The method further includes tightening the tool to engage the end of the wire guide, at least in part via pulling an attached control line, and withdrawing the tool from the patient through a second percutaneous entry point while engaged with the end of the wire guide, such that the wire guide simultaneously extends out of the patient at each of the first and second percutaneous entry points.

### BRIEF DESCRIPTION OF THE DRAWINGS

FIG. 1 is a side diagrammatic view of a tool for engaging and withdrawing a wire guide from a patient, according to the present disclosure;

FIG. 2 is a side diagrammatic view depicting one stage of a treatment procedure according to the present disclosure;

FIG. 3 is a side diagrammatic view depicting another stage of the treatment procedure;

FIG. 4 is a side diagrammatic view depicting yet another stage of the treatment procedure;

FIG. 5 is a side diagrammatic view depicting yet another stage of the treatment procedure; and

FIG. 6 is a side diagrammatic view at yet another stage of the treatment procedure.

### DETAILED DESCRIPTION

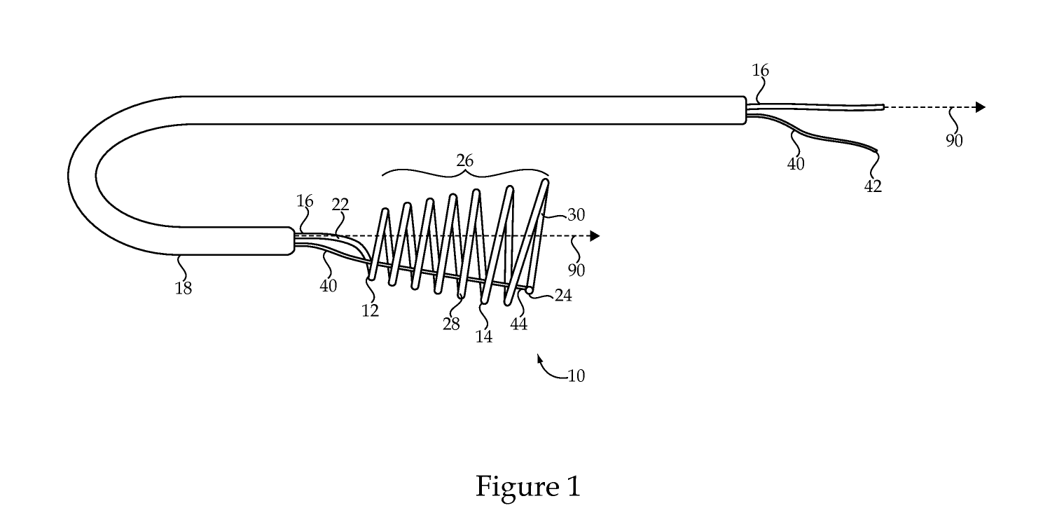

Referring to FIG. 1, there is shown a tool 10 for engaging and withdrawing a wire guide from a patient. Tool 10 is shown extending through a sheath 18 suitable for positioning intraluminally within the patient. Tool 10 includes an elongate body 12 having a deformable distal segment 14, and a rigid proximal segment 16 for sliding distal segment 14 into and out of sheath 18. Distal segment 14 includes a self-expanding bias such that distal segment 14 assumes a tortuous configuration defining a three-dimensional spatial envelope, in response to sliding out of intraluminal sheath 18, the significance of which will be apparent from the following description.

In FIG. 1, tool 10 is shown as it might appear where distal segment 14 is in a tortuous, rest configuration, having expanded via its self-expanding bias in response to sliding out of sheath 18. Sliding distal segment 14 into sheath 18, such as by manipulating one or both of proximal segment 16 and sheath 18, can apply a load opposing the self-expanding bias to adjust distal segment 14 from the tortuous configuration to a less tortuous and typically substantially linear configuration within sheath 18. Distal segment 14 may be elastically deformable between tortuous and linear configurations. A control line 40 is attached to distal segment 14 and configured to extend in parallel with proximal segment 16 through sheath 18, such that pulling control line 40 in a proximal direction deforms distal segment 14 in opposition to the self-expanding bias to tighten about a wire guide, as further described herein. Control line 40 may include a first end 42 which extends out of sheath 18 to enable a clinician to grasp control line 40 for manipulation thereof, and a second end 44 attached to distal segment 14. In a practical implementation strategy, control line 40 may include a wire, a thread such as a suture, or another elongate element having sufficient strength to deform distal segment 14 in opposition to its self-expanding bias without breaking control line 40. It should be appreciated that pulling control line 40 can deform distal segment 14 in a first manner, while sliding sheath 18 over distal segment 14 deforms it in a different manner. In each case, however, the induced deformation will tend to be opposed by the self-expanding bias, in other words the tendency of distal segment 14 to assume the tortuous configuration. Elongate body 12 may be formed from a suitable radiopaque metallic alloy, and may be differentially treated in distal segment 14 versus proximal segment 16 such as via heat treating or other known treatment techniques to impart the differing properties of deformability and rigidity, respectively, as well as to impart appropriate shape memory properties. Those skilled in the art will thus appreciate that distal segment 14 might be designed to have various tortuous shapes assumed when no load opposing its self-expanding bias is applied. Whether a given shape is tortuous may depend upon whether and how the given shape defines multiple twists or turns through space. A simple loop or a single hook would not likely be considered tortuous. A helical or spiral shape, in contrast, will likely be fairly considered tortuous. A shape having multiple twists or turns lying all in the same plane might be considered tortuous, but would not likely be fairly considered to define a spatial envelope, and certainly not a three-dimensional one. A grid, screen or net might be understood to have tortuous parts, but not likely fairly said to have a tortuous configuration overall.

Distal segment 14 may include a first end 22 attached to proximal segment 16, a second end 24 which is free apart from being attached to control line 40, and a plurality of turns 26 extending between first and second ends 22 and 24. Those skilled in the art will appreciate that body 12 may be formed from a single wire, thus first end 22 may be understood as that part of the single wire which has properties of distal segment 14, and adjoins a part of the single wire having properties of proximal segment 16. Control line 40 may be tied, attached via an adhesive, soldered, or connected to second end 24 via any other suitable mechanism.

As noted above, distal segment 14 includes turns 26 extending between first and second ends 22 and 24. In a practical implementation strategy, a number of turns 26 may be equal to at least three, and seven or more turns 26 are contemplated, as shown in FIG. 1. In the embodiment shown in FIG. 1, the spatial envelope defined by distal segment 14 is funnel shaped, and turns 26 include a proximal turn 28 defining a smaller radius, and a distal turn 30 defining a larger radius. Each of turns 26 is circumferential of a longitudinal axis 90 defined by proximal segment 16, and may be understood as axially advancing. In other words, paths of turns 26 through space advance relative to axis 90. Distal segment 14 also may project in a distal direction from proximal segment 16, and such that the funnel shaped spatial envelope opens in a distal direction.

It may also be noted from FIG. 1 that control line 40 is threaded through distal segment 14, and in particular defines a path which is inside all of turns 26. In other embodiments, control line 40 might be positioned inside less than all of turns 26, but will typically be positioned inside a number of turns 26 which is greater than one. The manner in which control line 40 is threaded through distal segment 14 may depend in part upon the shape assumed by distal segment 14 in its rest configuration. Distal segment 14 traverses a generally spiral path to define the funnel shaped spatial envelope in the FIG. 1 embodiment, however, alternatives are contemplated. Rather than a spiral path, in its rest configuration, distal segment 14 might traverse a helical path, or a path which reverses direction such as by initially extending from proximal segment 16 in a distal direction but then reversing direction to extend in a proximal direction.

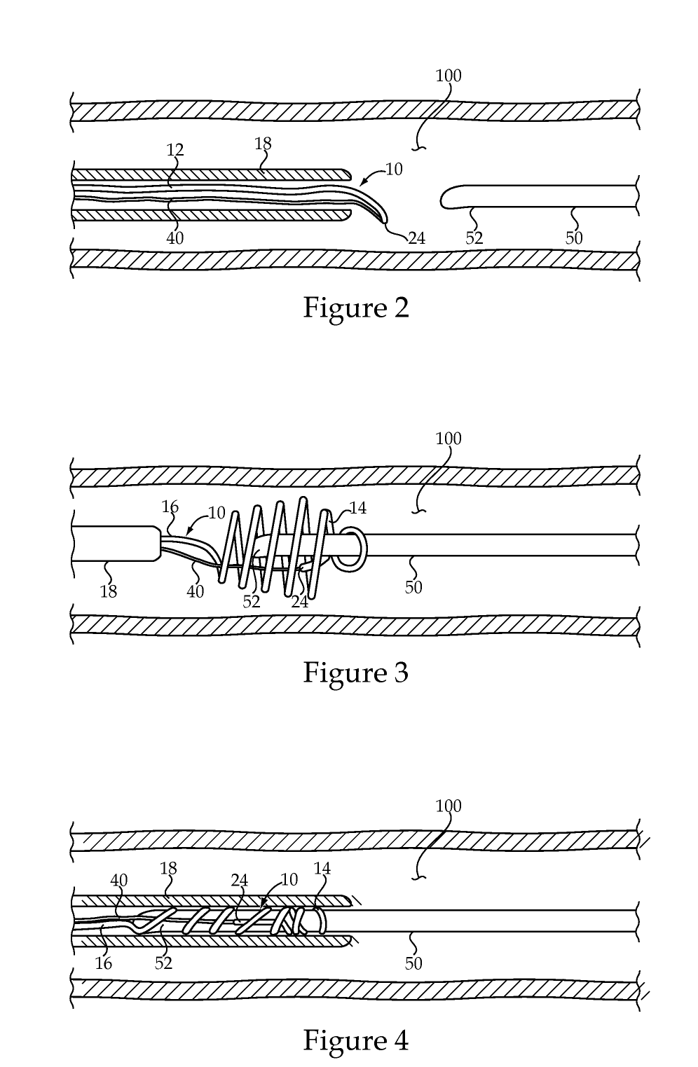

The shape assumed by distal segment 14 when used to engage with a wire guide may also depend upon the relative extent to which distal segment 14 has been slid out of sheath 18. Referring to FIG. 2, there is shown tool 10 extending part way out of sheath 18, in proximity to a wire guide 50 within a body lumen 100. Second end 24 has been advanced out of sheath 18 either by pushing proximal segment 16 while holding sheath 18 stationary, by holding proximal segment stationary while pulling back on sheath 18, or by a combination of these two actions. Referring to FIG. 3, there is shown tool 10 where an end 52 of wire guide 50 has been received within the spatial envelope defined by distal segment 14. In FIG. 3, control line 40 has been pulled in a proximal direction to deform distal segment 14 in opposition to its self-expanding bias to begin tightening distal segment 14 about end 52 of wire guide 50. Control line 40 has thus been pulled in a proximal direction through sheath 18 relative to proximal segment 16.

In FIG. 4, distal segment 14 has been returned into sheath 18 while tightened about wire guide 50. Tightening distal segment 14 in the manner described herein enables tool 10 to engage wire guide 50, generally establishing contact between distal segment 14 and wire guide 50 until sufficient frictional interaction between tool 10 and wire guide 50 exists to allow tool 10 to be withdrawn from the patient and pull wire guide 50 along with it. Establishing sufficient frictional interaction between tool 10 and wire guide 50 might occur solely via tightening of distal segment 14 about end 52 of wire guide 50, but could be assisted via a bias of sheath 18 acting upon distal segment 14 to further the tightening about wire guide 50. End 52, distal segment 14, or both, might also have a roughened surface texture or other features to enhance frictional engagement between the two components. It may also be noted from FIG. 4 that distal segment 14 has been deformed such that second end 24 has been pulled through the plurality of turns, and distal segment 14 is knotted about end 52 of wire guide 50. Returning to FIG. 2, it may be noted that control line 40 extends generally in parallel with body 12 through sheath 18. During adjusting distal segment 14 to its tortuous configuration, control line 40 may be captured inside a plurality of turns 26. Another way to understand this principle is that adjusting distal segment 14 to its tortuous configuration forms turns 26. Comparing FIGS. 2 and 3, it will be readily understood that forming turns 26 during adjusting distal segment 14 may capture control line 40 within turns 26 such that control line 40 becomes threaded through at least one, and typically all, of turns 26.

### INDUSTRIAL APPLICABILITY

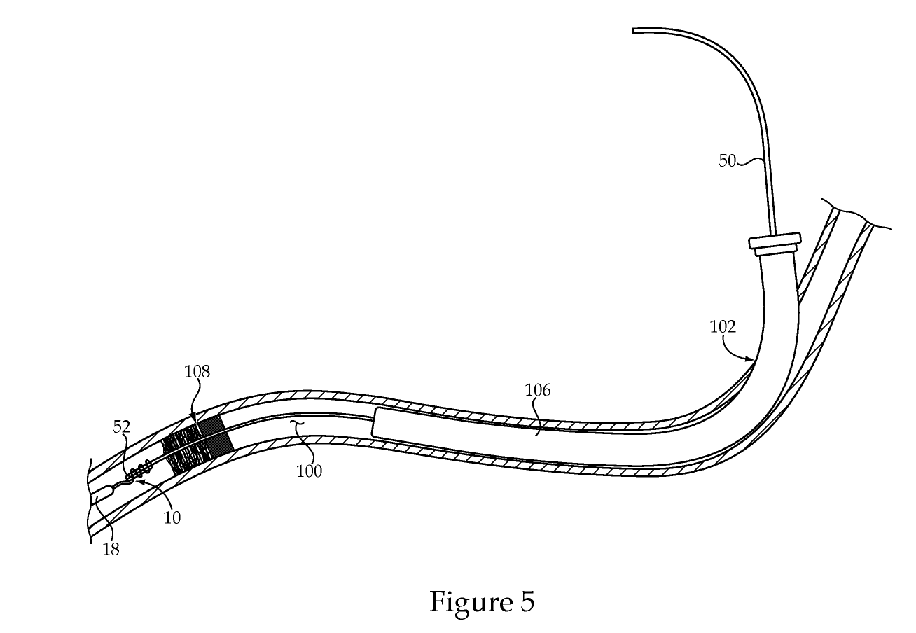

Referring to FIG. 5, there is shown tool 10 having been advanced from sheath 18, and where wire guide 50 has been advanced through body lumen 100 from a first percutaneous entry point 102 through an introducer sheath 106 or the like. End 52 of wire guide 50 has been received within the spatial envelope defined by tool 10 in the tortuous rest configuration of distal segment 14. In one embodiment, body lumen 100 may include an artery, and first percutaneous entry point 102 may be located below the patient's knee. In the FIG. 5 illustration, wire guide 50 has been advanced through lumen 100 such that it has crossed an occlusion 108. A flow of blood through lumen 100, if not completely blocked, may be generally from left to right in FIG. 5, such that wire guide 100 has been advanced through lumen 100 to cross occlusion 108 by being pushed in an upstream direction from entry point 102. In one practical implementation strategy, entry point 102 may include an entry point to the pedal artery (pedis dorsalis) of the patient, and may be located in the patient's foot or ankle, although the present disclosure is not limited. Moreover, it should be appreciated that the one or more occlusions to be treated may be located in arteries accessible via the pedal artery, and not in the pedal artery itself. Tool 10 has been slid out of sheath 18 at a location upstream occlusion 108, and is shown as it might appear just prior to being tightened about end 52 of wire guide 50. From the state depicted in FIG. 5, control line 40 may be pulled to tighten tool 10 about wire guide 50, and then tool 10 and wire guide 50 returned into sheath 18 in the manner described herein.

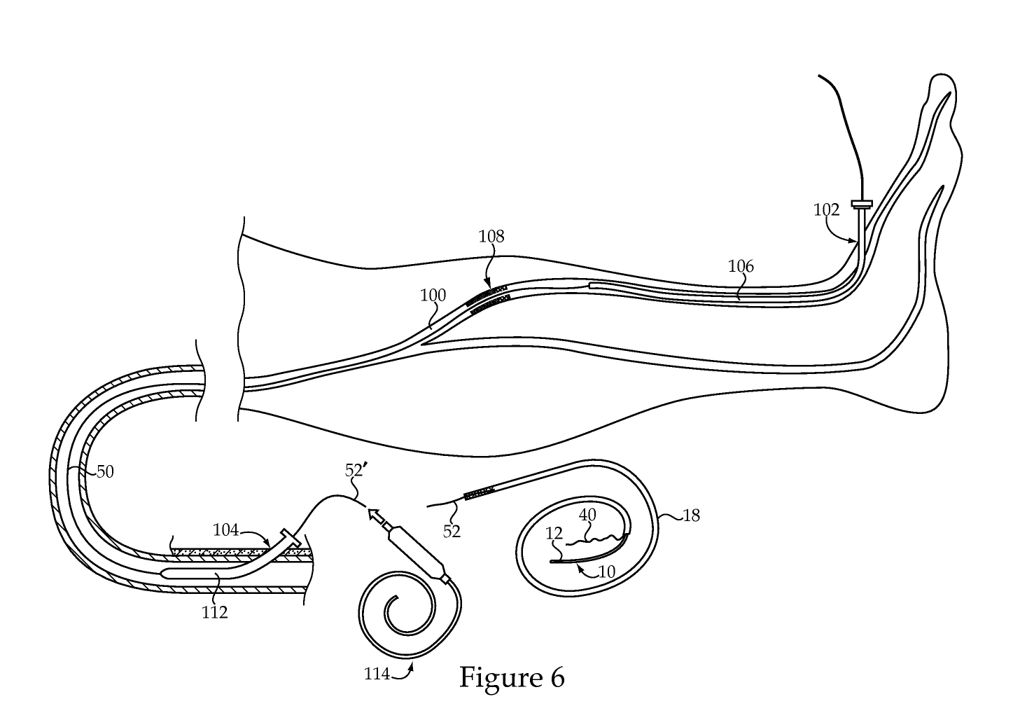

Referring to FIG. 6, there are shown tool 10 and sheath 18 having been withdrawn from the patient through a second percutaneous entry point 104. In particular, tool 10 and sheath 18 have been withdrawn through a second introducer 112 which extends through entry point 104 into a body lumen connecting with lumen 100, such as the same leg or opposite leg femoral artery. In FIG. 6, wire guide 52 has been cut to liberate end 52 and form a new end 52′ which extends out of entry point 104, such that wire guide 50 now simultaneously extends out of the patient at each of first and second entry points 102 and 104. A treatment device 114, such as a balloon angioplasty device, is shown as it might appear just prior to being slid over wire guide 50 to reach a site of occlusion 108 for treating the same.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A tool for engaging and withdrawing a wire guide from a patient comprising:
an elongate body including a deformable distal segment, and a rigid proximal segment for sliding the distal segment into and out of an intraluminal sheath;
the distal segment having a self-expanding bias such that the distal segment assumes a tortuous configuration defining a spatial envelope, in response to sliding out of the intraluminal sheath; and
a control line attached to the distal segment and configured to extend in parallel with the proximal segment through the intraluminal sheath, such that pulling the control line in a proximal direction tightens the distal segment in opposition to the self-expanding bias to engage a wire guide positioned within the spatial envelope.

2. The tool of claim 1 wherein the proximal segment defines a longitudinal axis, and wherein the distal segment further includes a first end attached to the proximal segment, a second end attached to the control line, and a plurality of axially advancing turns extending between the first and second ends.

3. The tool of claim 2 wherein each of the plurality of axially advancing turns is circumferential of the longitudinal axis.

4. The tool of claim 3 wherein the distal segment projects in a distal direction from the proximal segment.

5. The tool of claim 4 wherein the plurality of axially advancing turns includes a proximal turn defining a smaller radius, and a distal turn defining a larger radius.

6. The tool of claim 4 wherein the spatial envelope includes a funnel shape defined by the plurality of axially advancing turns, and wherein the funnel shape opens in the distal direction.

7. The tool of claim 2 wherein a number of the axially advancing turns is equal to at least three.

8. The tool of claim 7 wherein the elongate body is formed from a radiopaque metallic alloy, and the distal segment is elastically deformable between the tortuous configuration and a linear configuration.

9. The tool of claim 8 wherein the distal segment is in the tortuous configuration, and the control line is threaded through the distal segment.

10. The tool of claim 9 wherein the control line is positioned inside a number of the axially advancing turns greater than one.

11. A method of treating a patient comprising the steps of:
advancing a wire guide through a body lumen of the patient from a first percutaneous entry point;
receiving an end of the wire guide within a spatial envelope defined by a tortuous wire guide engagement and withdrawal tool;
tightening the tool to engage the end of the wire guide, at least in part via pulling an attached control line; and
withdrawing the tool from the patient through a second percutaneous entry point while engaged with the end of the wire guide, such that the wire guide simultaneously extends out of the patient at each of the first and second percutaneous entry points.

12. The method of claim 11 wherein the body lumen includes an artery, and the first percutaneous entry point is located below the patient's knee, and wherein the step of advancing further includes a step of crossing an occlusion in the artery at least in part by pushing the wire guide in an upstream direction from the first percutaneous entry point.

13. The method of claim 12 wherein the first percutaneous entry point includes an entry point to the pedal artery.

14. The method of claim 12 further comprising the steps of sliding the tool out of an intraluminal sheath at a location upstream the occlusion, and responsively adjusting the tool to a tortuous configuration via a self-expanding bias thereof

15. The method of claim 14 further comprising a step of capturing the control line inside a plurality of deformable turns of the tool during the step of adjusting, and wherein the step of tightening includes pulling the control line through each of the plurality of deformable turns.

16. The method of claim 14 further comprising a step of returning the tightened tool into the intraluminal sheath, and wherein the step of withdrawing further includes withdrawing the tool while positioned within the intraluminal sheath.

17. The method of claim 16 wherein the spatial envelope includes a funnel shaped spatial envelope defined by the plurality of deformable turns, and the step of receiving further includes receiving the end of the wire guide into an open end of the funnel shaped spatial envelope.

18. The method of claim 17 wherein the step of tightening further includes pulling an end of the tool through the plurality of deformable turns toward the second percutaneous entry point.

19. The method of claim 12 further comprising a step of guiding a treatment device from the second percutaneous entry point to a site of the occlusion via the wire guide.
